(19) United States
(12) Patent Application Publication
(10) Pub. No.: US 2006/0155158 A1
(43) Pub. Date: Jul. 13, 2006

(54) Percutaneously introduced blood pump and related methods

(76) Inventor: Walid Nagib Aboul-Hosn, Fair Oaks, CA (US)

Correspondence Address: Jonathan Spangler, 2875 Kalmia Place, San Diego, CA, 92104, US

(21) Appl. No.: 10/521,044

(22) PCT Filed: Jun. 11, 2003
(86) PCT No.: PCT/US03/18638

§ 371 Date: Dec. 7, 2005

Related U.S. Application Data
(60) Provisional application No. 60/388,113, filed on Jun. 12, 2002.
(60) Provisional application No. 60/431,174, filed on Dec. 4, 2002.

(51) Int. Cl. A61M 1/12

(52) U.S. Cl. 600/16

(57) ABSTRACT

The present invention involves a blood pump having a pump housing with an internally disposed rotor that collectively provide a dual inflow capability which prevents thrombus formation and reduces hemolysis during use.

### CROSS-REFERENCES TO RELATED APPLICATIONS

The present application is an International Patent Application of and claims the benefit of priority from commonly owned and co-pending U.S. Provisional Patent Application Ser. Nos. 60/388,138 (filed Jun. 11, 2002) and 60/431,174 (filed Dec. 4, 2002), the entire contents of which are hereby expressly incorporated by reference into this disclosure as if set forth fully herein.

### BACKGROUND OF THE INVENTION

I. Field of the Invention

The present invention relates generally to a system for assisting the heart and, more particularly, to a pumping system and related method for supplementing the circulation of blood through the patient using a minimally invasive procedure.

II. Description of Related Art

Over the years, various types of percutaneously introduced blood pumps have been developed for the purpose of augmenting or replacing the blood pumping action of damaged or diseased hearts. Such blood pumps may be positioned within the heart of the patient (so-called “intracardiac blood pumps”) or may be positioned within the associated vasculature of the patient (so-called “intravascular blood pumps”). Such percutaneously introduced blood pumps have experienced proliferated growth and attention in that they are capable of supplementing or replacing the circulation of blood through the patient using minimally invasive techniques (eliminating the trauma of an open procedure), and minimize the need to route the blood outside the patient (reducing trauma to the blood).

Although generally advantageous for these reasons, among others, the percutaneously introduced blood pumps of the prior art nonetheless suffer from various drawbacks. One drawback involves the potential for thrombus formation. More specifically, the prior art percutaneously introduced blood pumps are characterized in that they have a single blood inflow region. Based on this single direction of blood inflow, the potential exists that blood can stagnate on the distal or downstream end of the rotor, which may precipitate thrombus formation over time.

A still further drawback with prior art percutaneously introduced blood pumps relates to hemolysis. That is, the single blood inflow feature of prior art percutaneously introduced blood pumps effectively limits the amount of blood that can enter the blood pump. Based on this limitation, the rotor has to be operated at a high rate of speed to achieve high flow rates, and high rotor speeds are known to increase the extent to which the blood cells become damaged over time.

The present invention is directed at eliminating, or at least reducing the effects of, the above-described problems with the prior art.

### SUMMARY OF THE INVENTION

The present invention overcomes the limitations of the prior art by providing a pumping system capable of being percutaneously introduced into a patient to augment or replace the pumping capacity of the patient's heart. More specifically, this is accomplished by equipping the blood pump of the present invention with a pump housing with an internally disposed rotor that collectively provide a dual inflow capability which prevents thrombus formation and reduces hemolysis during use. As used herein, “dual inflow” is defined as having blood enter the blood pump from at least two directions. Thrombus prevention is accomplished in that the dual inflow feature causes the entering blood to wash over the rotor in at least two directions, which minimizes (if not eliminates) the extent to which the blood can stagnate within the blood pump. Hemolysis reduction is accomplished in that the dual inflow feature allows the blood pump to be operated at lower speeds without adversely affecting (that is, lowering) the flow rate of blood. This is based on the increased supply of blood that can enter the blood pump via the multiple inlets. By reducing the speed at which the rotor is operated, the resulting hemolysis (that is, damage to the blood cells) is lower than would otherwise be possible with a single fluid inlet, which would require the rotor to be operated at a higher speed to produce an equivalent flow rate.

With the ability to be applied within a minimally invasive procedure, the present invention significantly improves the applicability of such treatment to a wider group pf patient at a much reduced morbidity and mortality. An ancillary but important benefit of the present invention is the ability to apply the present invention in such a way as to also reduce the pumping load on the heart, thereby potentially permitting the heart to recover during use.

The present invention also achieves a variety of objectives, several of which are set forth below by way of example only. One such object is to have a dual pumping system within one diameter, therefore significantly increasing the flow capacity of the device. Theoretically, the dual inflow pumps should double the device total flow when compared to a similar device of similar diameter.

Another object of this invention is to have an intracardiac system that comprises a rotary pump configured to be fully implanted inside the patient's heart with an internal battery implanted subcutaneously in the thoracic or abdominal area. In addition, a transcutaneous coil is implanted in the proximity of the implanted battery to allow transcutaneous power transmission to periodically charge the internal battery.

The intracardiac system of the present invention preferably comprises at least one rotary pump configured to pump blood through the patient at cardiac rates. Importantly, the preferred pump for the present invention pumping system is one that requires a relatively low amount of energy input, when compared to prior art pumps designed to pump at cardiac rates. A magnetically suspended rotary pump is known to offer the highest efficiency for such application since friction between rotary and static components is nearly eliminated.

The present invention retains the rotor in optimal radial and axial position at all times. The rotor is suspended radially, and magnetically biased in one axial direction. Axial position is held by at least a single-point, preferably two points, contact thrust bearing, and all thrust forces on the rotor, including those resulting from the hydrodynamic interaction of the impeller with the blood, are maintained below the force required to displace the thrust bearing from contact. Furthermore, when thrust balancing of the pump impellers is used, force on the thrust bearing is minimized. Since the thrust-bearing contact point is at the center of rotation, surface friction, wear, and heat generation are also minimized. Also, the thrust bearing point is located in a high-flow position for sufficient washing to prevent thrombus accumulation.

The magnetic bearing of the present invention may also incorporate mechanical radial position limiters to maintain alignment of the rotor in nearly centered radial position if transient forces momentarily overcome the magnetic radial bearing capacity. Such mechanical limiters might be part of the thrust bearing and take the form of mechanical radial bearings having a large enough radial clearance between the stationary and rotating members so that in usual operation they do not support the radial load and do not wear. The axial thrust and radial limiter bearing may be composed of wear-resistant materials, such as ceramic, or may utilize wear-resistant inserts, and the rotor tip in proximity to the thrust bearing may also be fabricated of wear-resistant materials. Thus, even with occasional mechanical contact, no galling of the surfaces or other damage to the pump will occur.

Another object of the present invention is to provide a miniature and extremely durable blood pump for highly reliable long-term use.

Another object of the present invention is to provide a blood pump utilizing nearly complete magnetic suspension of a rotating pump impeller combined with the most minimal mechanical thrust-bearing component possible.

Another object of the present invention is to provide a dual-inlet artificial heart capable of operation at approximately half the rotational speed of a single-inlet device of comparable diameter.

Another object of the present invention is to provide a radially magnetically suspended blood-pump rotor having near perfect thrust balancing to permit effective operation with minimal thrust-load variation under pulsatile flow conditions.

Another object of the present invention is to provide a radially magnetically suspended blood-pump rotor having mechanical bearing safety backup capability during conditions of transient magnetic bearing overload.

Another object of the present invention is to provide an outflow cannula fluidly coupled to the pump, to direct blood from the pump to a primary blood vessel, such as the aorta when the system is used for left ventricular assist. The cannula traverses a heart valve, such as the aortic valve when used as a left ventricular assist device. The pump inflow is direct from the heart chamber the pump is implanted in.

Another object of the present invention is to provide a method where the entire system of the present invention is implanted through a major peripheral vein without the need for major invasive surgery. For example, the pump may be implanted through the groin area, and advanced using standard techniques for tracking the device inside an introducer sheath to its final destination. It is contemplated that the device will be advanced through the atrial septum when it is intended to function as a left ventricular assist device.

Another object of the present invention is to provide a blood pumping system that is introduced through the venous system and advanced to the left atrium, wherein a centrifugal or axial rotary blood pump is positioned into the left atrium and the pump's outflow cannula is advanced through the aortic valve into the aorta. The blood pump basically remove blood from the left atrium and deliver it to the aorta, therefore bypassing the left ventricle and reducing the workload required by the left ventricle.

Another object of the present invention is to provide a blood pumping system that is introduced through the venous system and partially advanced to the left atrium, wherein a centrifugal or axial rotary blood pump is positioned into the left atrium, an electric motor is deployed in the right atrium and provides rotational motion to the pump across the atrial septum, and the pump's outflow cannula is advanced through the aortic valve into the aorta. The blood pump basically removes blood from the left atrium and delivers it to the aorta, therefore bypassing the left ventricle and reducing the workload required by the left ventricle. Therefore, the blood pump size is minimal while the motor size is not constrained since it could use the space in the right atrium.

Another object of the present invention comprises a method and device for accessing the left atrium or left ventricle of the heart by utilizing an introducer and a guide catheter in which a needle assembly is advanced axially. The guide catheter is advanced through a peripheral vein and advanced to the atrial or ventricular septum under fluoroscopic guidance. A needle assembly is used to perforate the septum before advancing the catheter through the septum. An introducer is advanced over the catheter (and can slide thereover) and is inserted into a blood vessel to the left atrium. Once the introducer has been advanced into the left atrium the needle assembly and the catheter can be easily withdrawn. Thus, a device could be advanced through the introducer to the left atrium safely, quickly, and without compromising sterility.

Another object of the present invention comprises a method and device for accessing the left atrium or left ventricle of the heart by utilizing a succession of introducers with different tip curvature that the insertion of one introducer inside the other would result in complex cumulative curvature at the tip of the assembled introducers. The successive curvature would allow the advancement of complex geometry in a complex structure that is not achievable by one introducer with similar complex tip geometry. The present method and device for accessing the left atrium or left ventricle of the heart comprise a method and a device for accessing the left atrium or left ventricle of the heart by utilizing at least two introducers and a catheter assembly in which a needle assembly is advanced axially. The guide catheter is advanced through a peripheral vein and advanced to the atrial or ventricular septum under fluoroscopic guidance. A needle assembly is used to perforate the septum before advancing the catheter. Then an introducer with curved tip is advanced over the catheter (and can slide thereover) and is inserted into a blood vessel to the left atrium. For example, the first introducer tip curvature would result in pointing the introducer toward the left ventricle. Following, an introducer with a different curved tip is advanced inside the first introducer and is inserted into the left ventricle. For example, the curvature of the second introducer would point the introducer into the aortic valve; therefore any device advanced through the two introducers will lead to the aortic valve automatically. Once the introducers have been advanced into the left ventricle the catheter can be easily withdrawn. Thus, a device could be advanced through the introducer to the left ventricle or aorta safely, quickly, and without compromising sterility.

Another object of the present invention is to provide a method where the a dual guide wire system that consist of two separate guide wires with a magnetic tip, wherein one guide wire is advanced through the arterial system to the left ventricle and a second guide wire is advanced though the venous system and through the atrial septum to the left ventricle. The two guide wires join in the ventricle using magnetic attraction between the two magnets positioned at each guide wire tip. A device is attached to the proximal end of the guide wire inserted through the venous side is pulled through the venous system by pulling the guide wire inserted into the arterial system, therefore pulling the pump into the arterial system from the venous system across the atrial septum.

Another object of the present invention is to provide an alternative outflow cannula fluidly coupled to the pump, to direct blood from the pump to a primary blood vessel, such as the aorta when the system is used for left ventricular assist. The cannula traverses atrial septum, the right atrium and the aorta wall in proximity to the right atrium. The pump inflow is direct from the heart chamber the pump is implanted in.

Another object of the present invention is to provide a method where the alternative system, which is described in the previous paragraph, of the present invention is implanted through a major peripheral vein without the need for major invasive surgery. For example, the pump may be implanted through the groin area, and advanced using standard techniques for tracking the device inside an introducer sheath to its final destination. It is contemplated that the device will be advanced through the atrial septum when it is intended to function as a left ventricular assist device. The alternative outflow cannula will be advanced onto the aorta through an opening created in the wall section of the right atrium adjacent to the ascending aorta.

Another object of the present invention comprises a method and device for reducing the left or right ventricle volume by the simultaneous use of the pumping system mentioned above and a “containment” device intended to physically contain and decrease the ventricle volume. The containment device could be similar to the CorCap™ Cardiac Support Device, developed by Acorn cardiovascular Inc of St Paul, Minn. USA, with one main difference; the “containment” device would be dynamic. In other words, the containment device would actively and continually force the ventricle's volume to decrease.

Another object of the present invention comprises a method and device for reducing the left or right ventricle volume by the simultaneous use of the pumping system mentioned above in conjunction with injections of engineered or autologous tissue into the myocardium to help regenerate a healthy myocardium. In essence, the heart load is decreased significantly by the use of the pumping system while the injected tissue is allowed to establish a healthy myocardium. This myocardium is allowed to grow while the heart load and heart volume is decreased; therefore, allowing the remodeling of heart hypertrophy.

### BRIEF DESCRIPTION OF THE DRAWINGS

FIG. 1 is a schematic view of a first embodiment of the present invention shown disposed within a patient's heart;

FIG. 2 is a schematic view of the first embodiment of the present invention with cross-sectional views at different levels there along;

FIGS. 3 (a) and (b) are cross-sectional views (longitudinal and taken along line C-C, respectively) of the first embodiment of the present invention shown in FIG. 2;

FIG. 4 (a) is a longitudinal cross-sectional view of the pump head;

FIGS. 4 (b) and (c) are cross-sectional views of the pump head taken along lines B-B and C-C, respectively, of FIG. 4(a);

FIGS. 5 (a) and (b) are schematic and cross-sectional views of the pump rotor and pump housing, respectively, showing magnet placement;

FIGS. 6 (a) and (b) are cross-sectional and schematic views, respectively, illustrating the pump thrust bearings and magnet placement of the pump rotor and pump housing;

FIGS. 7 (a)-(c) are various cross-sectional views of a second embodiment of the present invention;

FIG. 7 (d) is a schematic view of the second embodiment of the present invention shown disposed within a patient's heart;

FIGS. 8 (a)-(c) are schematic views of an introducer system of the present invention suitable for introducing the pump system of the present invention into a patient's heart;

FIGS. 9 (a)-(c) are schematic views of the first, second, and third steps, respectively, in using the introducer of FIGS. 8(a)-(c) to introduce the blood pump of the present invention into the patient's heart as shown in FIG. 1;

FIGS. 10 (a) and (b) are schematic and exploded views, respectively, of a coupling mechanism to aid in the insertion of the blood pump shown in FIG. 1 into the patient;

FIG. 11 is a schematic view of the first embodiment of the present invention in use as part of an overall system;

FIG. 12 is a schematic view of the first embodiment of the present invention, shown applied to right and left side of a patient's heart;

FIGS. 13 (a)-(c) are schematic views of the first step, second, and third step in inserting the blood pump of the present invention (shown in FIG. 1 and/or FIG. 7);

FIG. 14 is a schematic view of a third embodiment of the present invention shown disposed within a patient's heart;

FIG. 15 is a schematic view of a fourth embodiment of the present invention shown disposed within a patient's heart;

FIG. 16 is a schematic view of a fifth embodiment of the present invention shown disposed within a patient's heart;

FIG. 17 is a schematic view of a guide wire system of the present invention shown extending through the patient's vasculature and heart;

FIGS. 18 (a)-(c) are schematic view of the first, second, and third steps in inserting the blood pump of the present invention (shown in FIG. 1 or FIG. 7) via the guide wire system shown in FIG. 17; and

FIG. 19 is a schematic view of the blood pump of the present invention used in conjunction of a “containment” device disposed about part of a patient's heart.

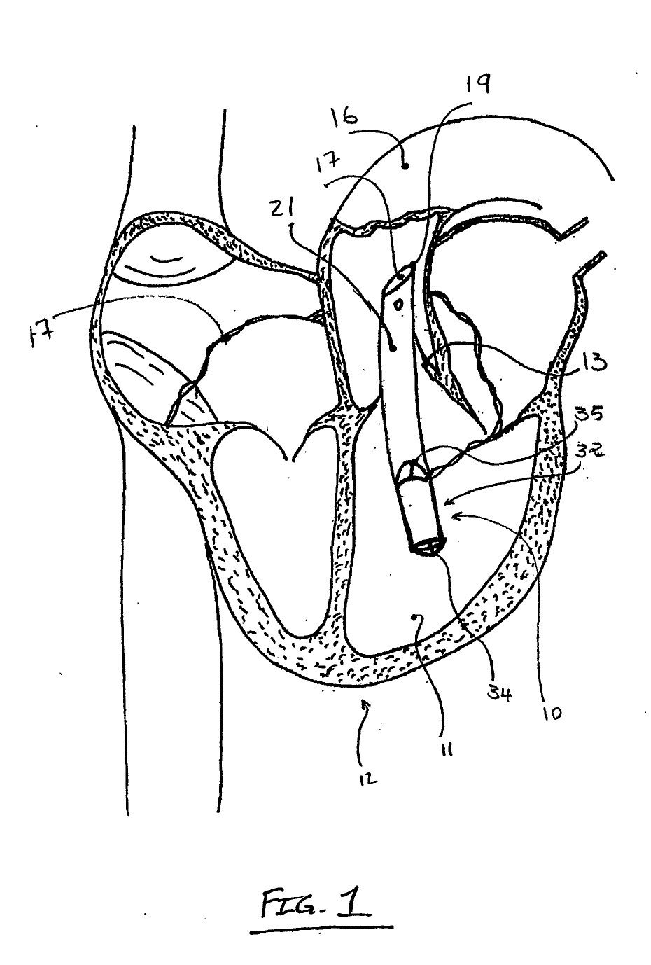

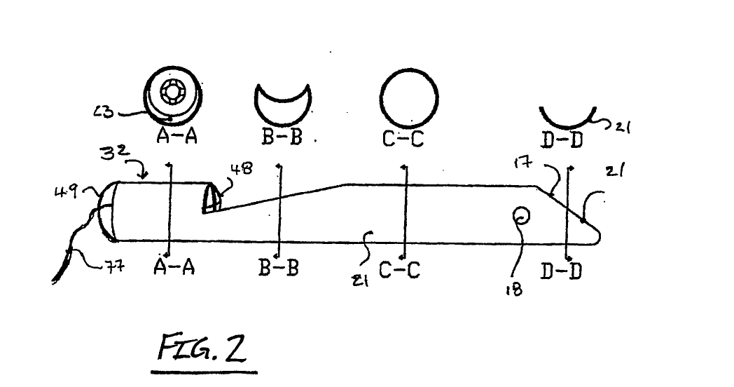

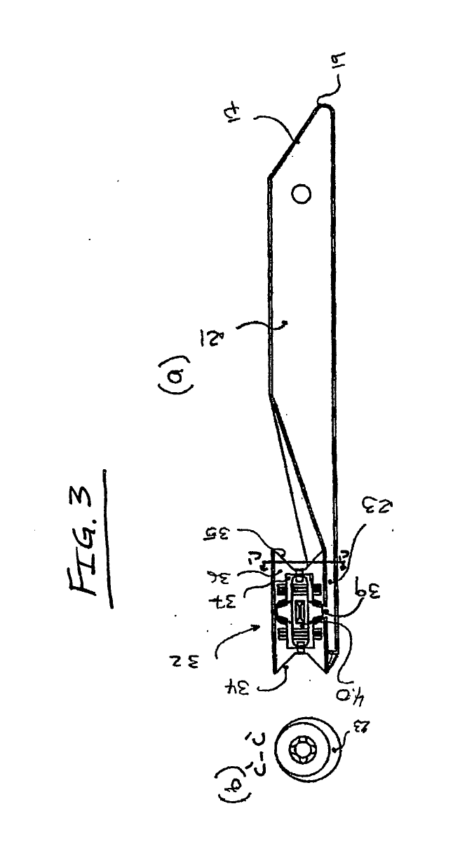

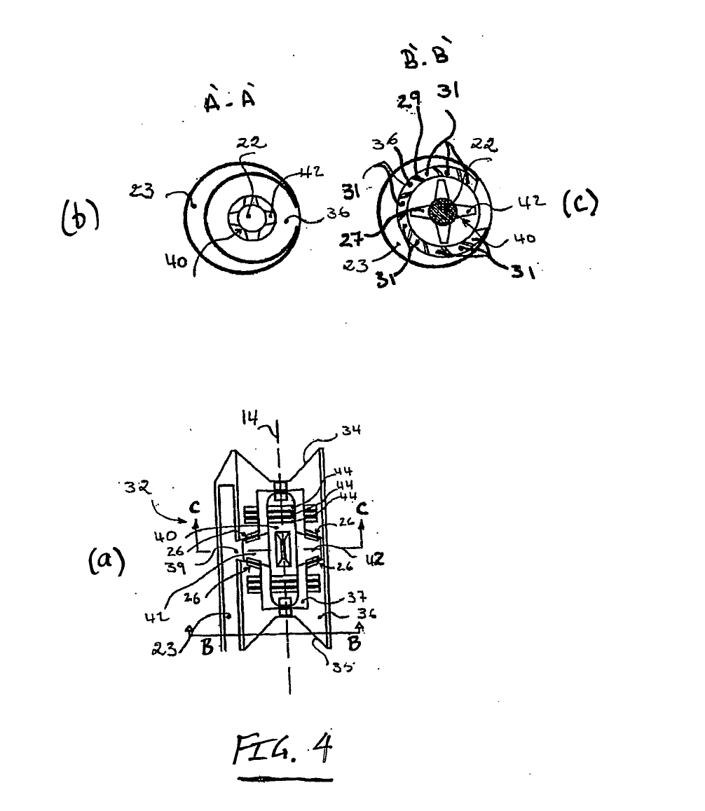

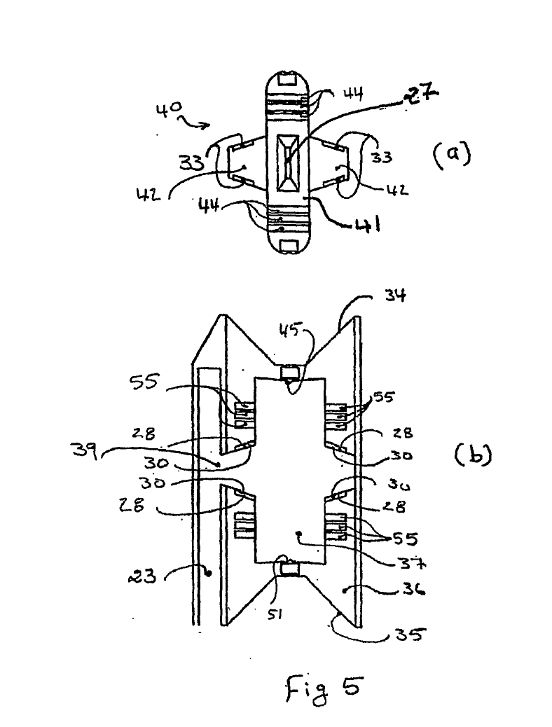

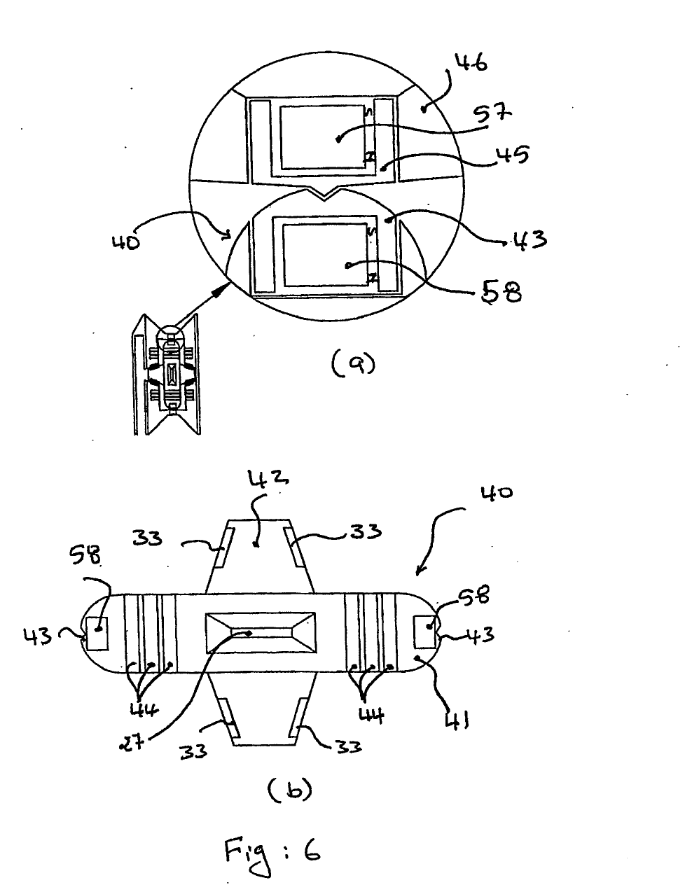

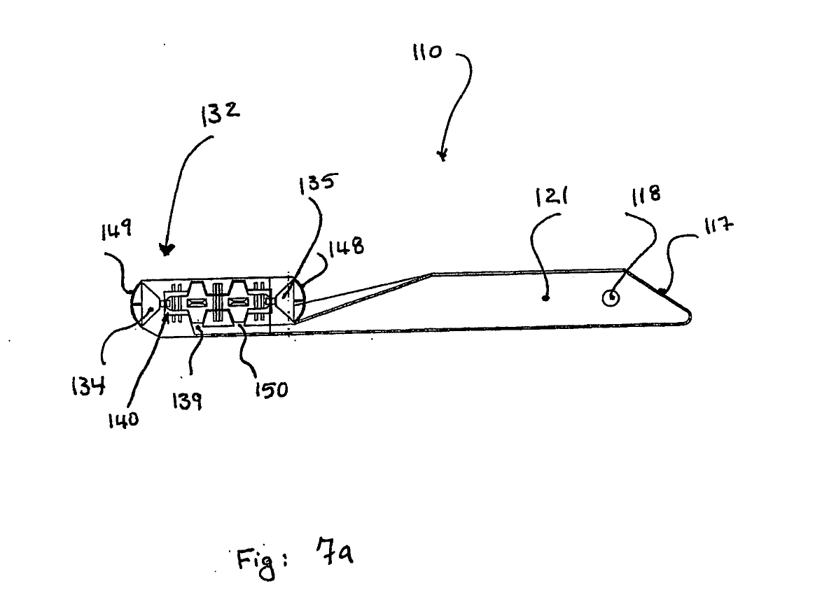

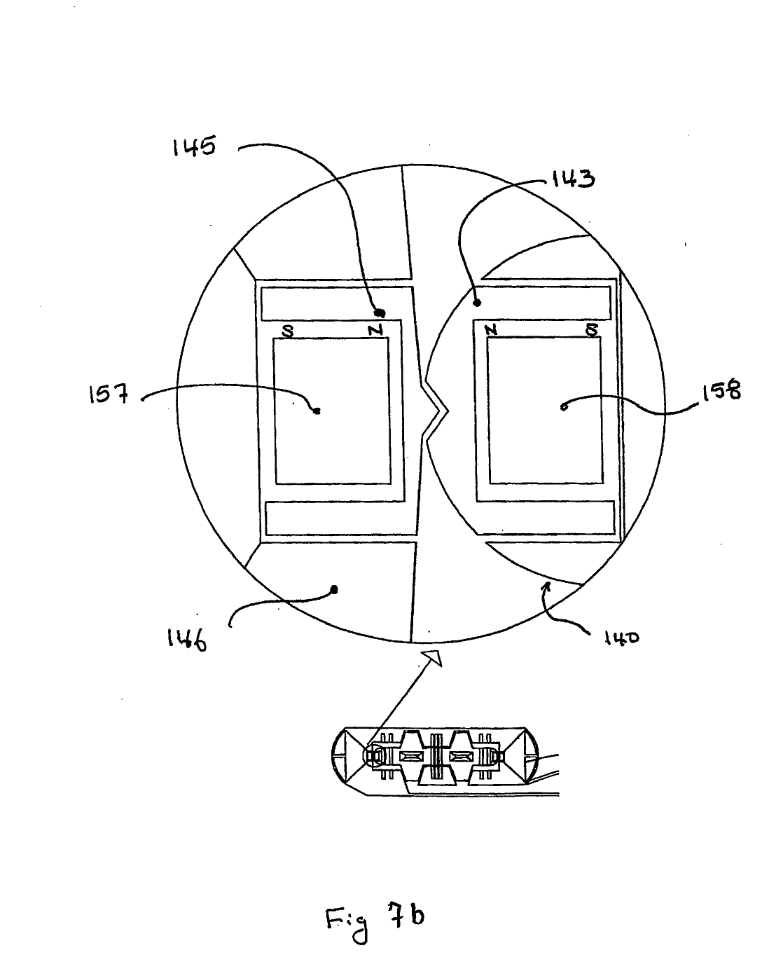

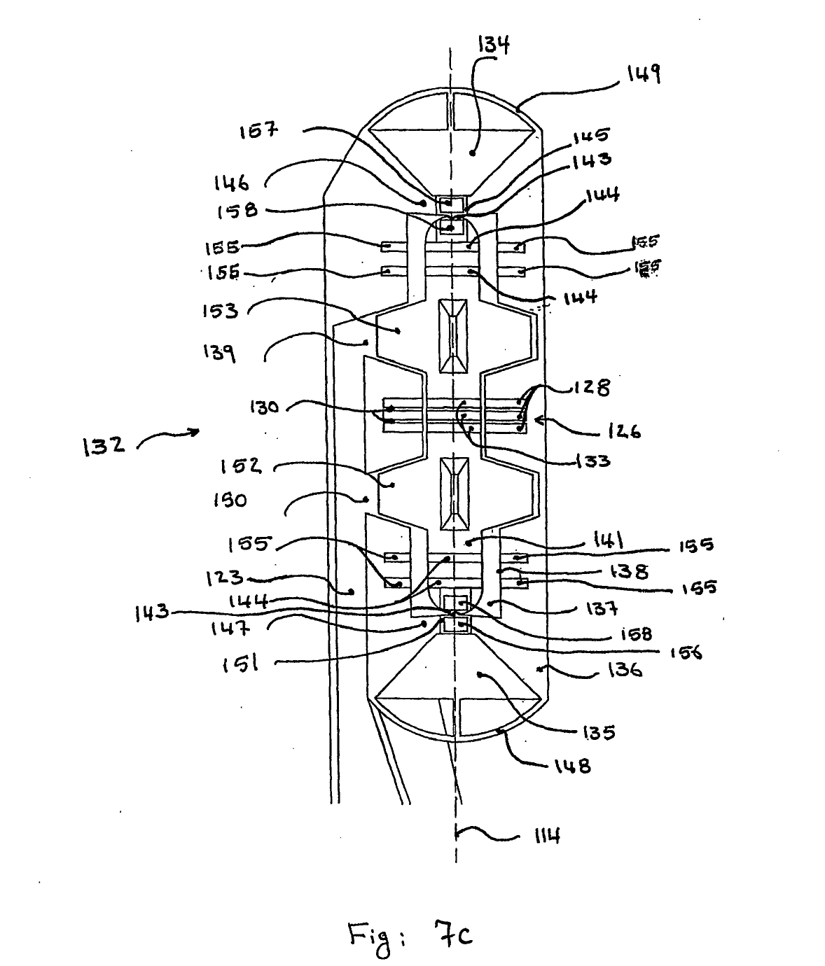

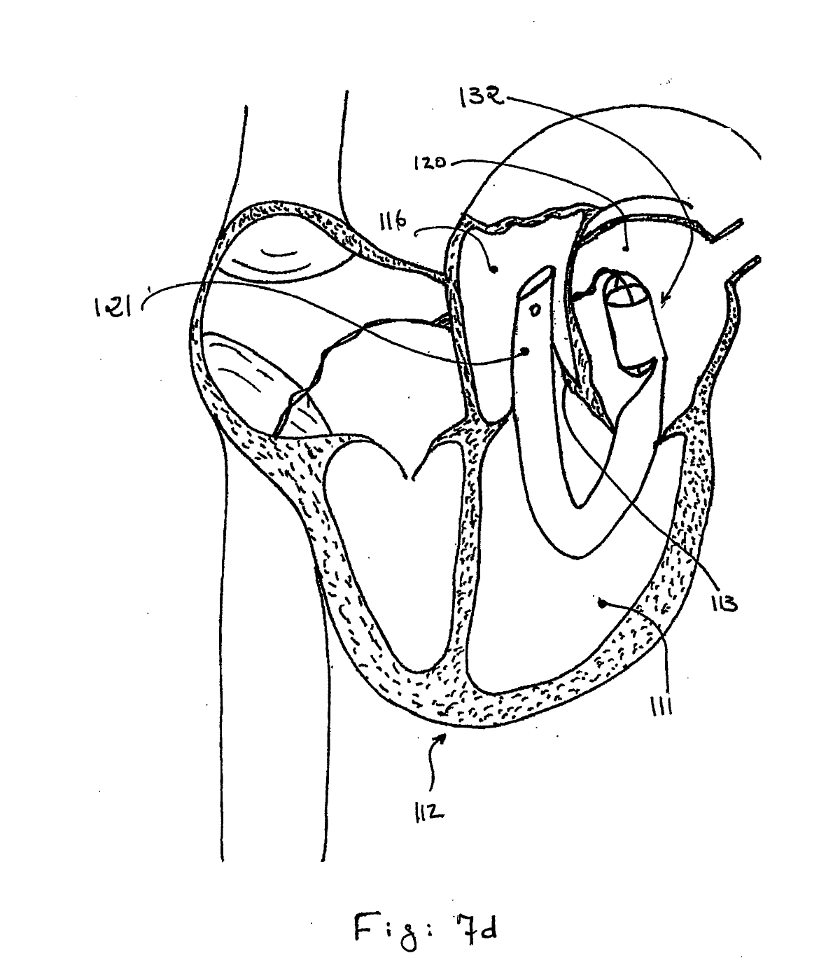

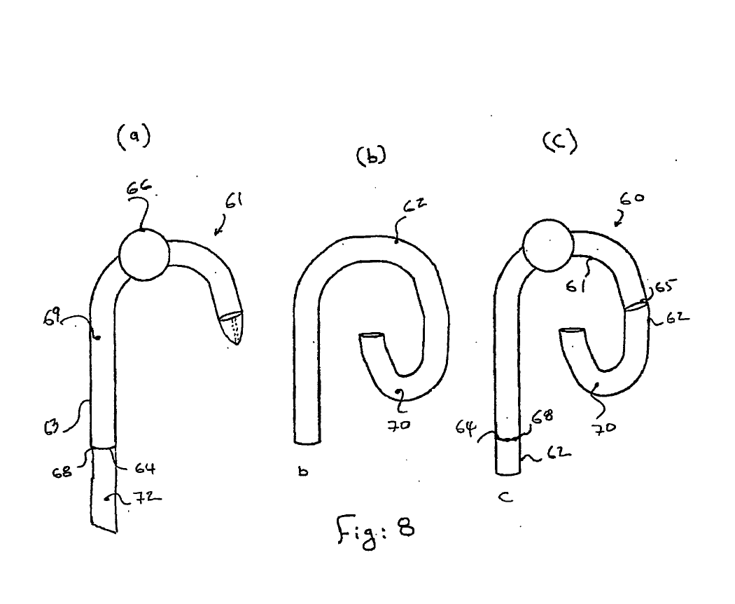

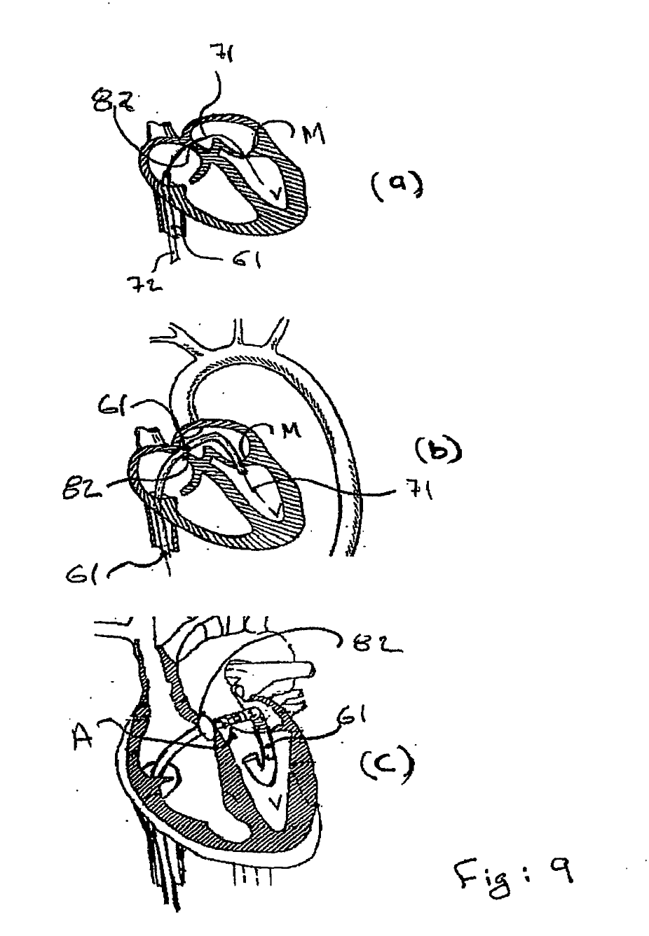

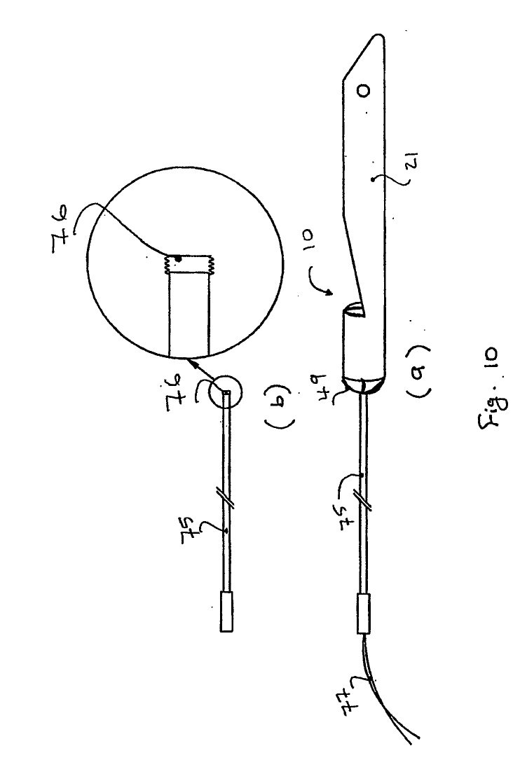

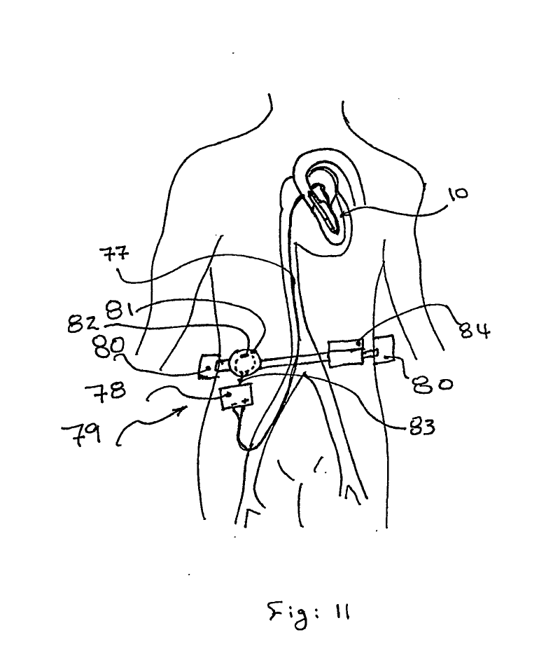

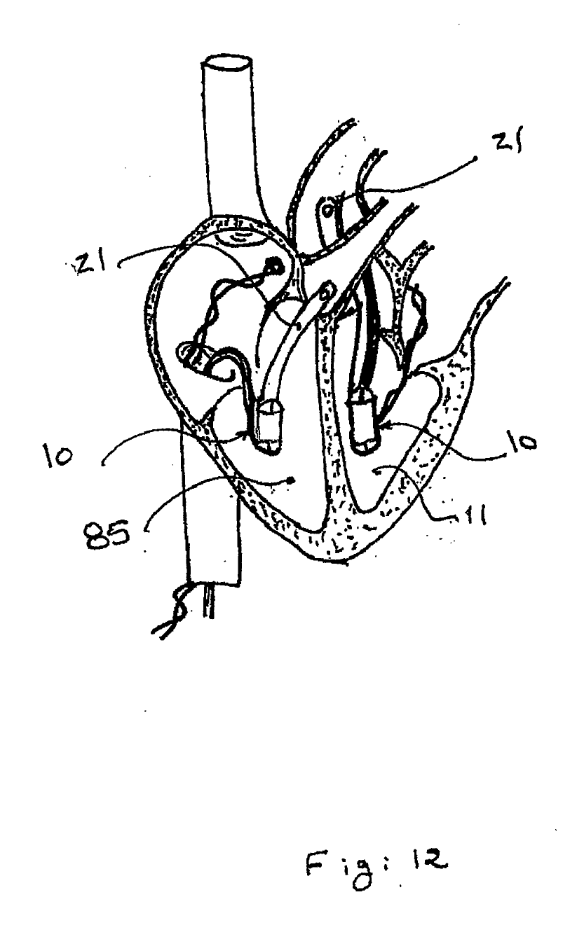

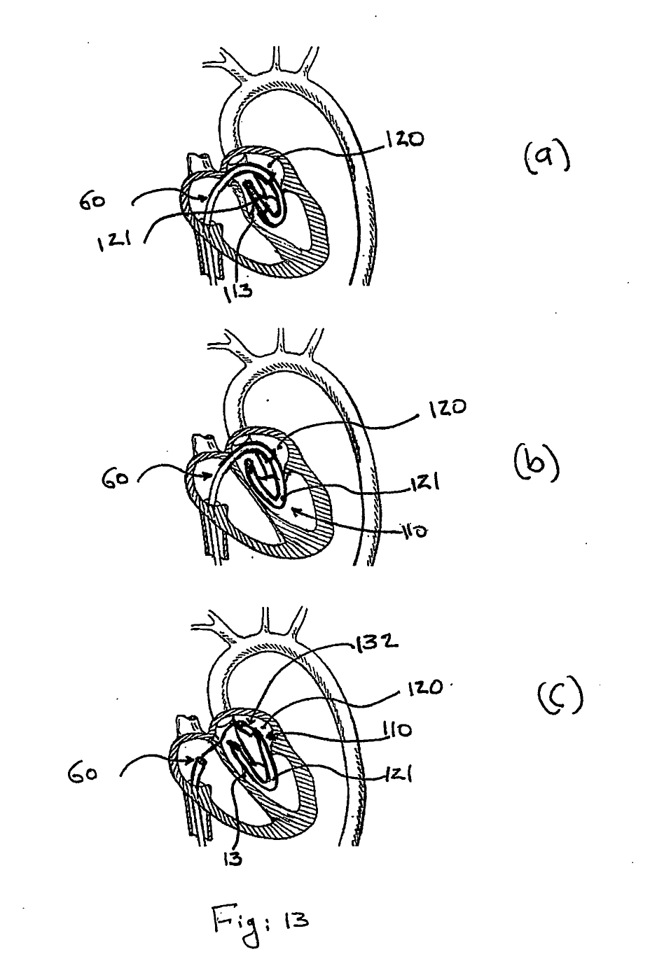

### DESCRIPTION OF THE SPECIFIC EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The systems disclosed herein boast a variety of inventive features and components that warrant patent protection, both individually and in combination.

The present invention involves a blood pump capable of being percutaneously introduced into a patient to augment or replace the pumping capacity of the patient's heart. As will be described below, the blood pump of the present invention includes a pump housing with an internally disposed rotor that collectively provide a dual inflow capability which prevents thrombus formation and reduces hemolysis during use. As used herein, “dual inflow” is defined as having blood enter the blood pump from at least two directions. Thrombus prevention is accomplished in that the dual inflow feature causes the entering blood to wash over the rotor in at least two directions, which minimizes (if not eliminates) the extent to which the blood can stagnate within the blood pump. Hemolysis reduction is accomplished in that the dual inflow feature allows the blood pump to be operated at lower speeds without adversely affecting (that is, lowering) the flow rate of blood. This is based on the increased supply of blood that can enter the blood pump via the multiple inlets. By reducing the speed at which the rotor is operated, the resulting hemolysis (that is, damage to the blood cells) is lower than would otherwise be possible with a single fluid inlet, which would require the rotor to be operated at a higher speed to produce an equivalent flow rate.

Although the blood pump of the present invention is described herein mainly in terms of an intracardiac application (that is, disposed within the heart), it is to be readily appreciated that it may find application in any number of areas within the patient's circulatory system without departing from the scope of the present invention. Moreover, the dual inflow feature of the present invention is described below as being carried out by providing blood inlets at opposing ends of the pump housing such that, in use, the rotor draws the blood through the two blood inlets in a generally axial direction relative to the rotor and directs it out at least one blood outlet disposed at some point in between the opposing ends of the pump housing. It will be appreciated, however, that the direction of the dual inflow need not be generally axial, but rather may be any suitable direction (e.g., skewed) to cause at least two distinct flows washing over some or all of the rotor.

The blood pump of the present invention described herein is sized to pump blood at rates comparable to the flow rate of an average healthy heart. That is, it may be sized and configured to discharge blood at volumetric flow rates anywhere in the range of 1 to 8 liters per minute, depending upon the application desired and/or the degree of need for heart assist. For example, for a patient experiencing advanced congestive heart failure, it may be preferable to employ a pump that has an average flow rate of 4.5 to 6 liters per minute. In other patients, particularly those with minimal levels of heart failure or patients that had recovered considerably from heart failure, it may be preferable to employ a flow rate of 3 liters per minute or less.

With reference to FIG. 2, a first embodiment of the present invention system 10 is shown (by way of example only) implanted in an ailing heart 12 with pump 32 positioned in the left ventricle 11 and outflow cannula 21 placed across the aortic valve 13 in the aorta 16. In accordance with the preferred embodiment of the present invention, and with particular attention being directed to FIGS. 2 and 3 of the drawings, the pump generally designated 32 comprises a housing 36, the interior of which defines pumping chamber 37. Pump 32 having dual inlets, distal inlet 34 and proximal inlet 35, and an outflow cannula 21 to provide a conduit for blood energized by pump 32 to cross from the left ventricle 11 into aorta 16 across aortic valve 13. Pump 32 is preferably a centrifugal rotary pump; although either an axial type or a centrifugal type could be interchanged. In either case, pump 32 is sufficiently small to be implanted percutaneously through a main artery or vein such as the femoral vein in the groin area of the patient, without the need for major invasive surgery.

With attention being directed to FIGS. 3 and 4 the inner periphery 38 of housing 36 is the outer periphery of the chamber 37. As is clear from the views of FIGS. 2 and 3, housing 36 and chamber 37 shares a central axis that extends along axis 14 as set forth in FIG. 4. Housing 36, and accordingly chamber 37, is provided with a pair of inlet, distal inlet 34 and proximal inlet 35, along with outlet port 39. Inlet ports 34 and 35, collectively, define the inlets to chamber 37, while outlet port 39 define the outlet. The inlet ports 34 and 35 are arranged coaxially with the chamber, that is, along axis 14, with the inlet ports being arranged in oppositely disposed relationship to chamber 37. One or more inflow stators (struts) may be provided projecting into the two pump inlets to act as a hydraulic stator to condition blood flow into pump 32. In addition, two protective cages, proximal protective cage 48 and distal protective cage 49, protect surrounding tissue from entering the pump or from pump suction against a relatively flat surface. Outlet port 39 is arranged medially of the inlet ports, and is, as indicated, disposed generally circumferentially. Outlet port 39 is formed from a multitude of outflow channel 31 (as shown in FIG. 4d), which empties into discharge chamber 23. Successive outflow channel 31 are separated by hydraulic stator 29, which is designed to direct blood from chamber 37 into discharge chamber 23. Alternatively, outlet port 39 could be a tangential opening (not shown) between chamber 37 and discharge chamber 23 that approach the design of the volute of a centrifugal pump. Volute design is, of course, commonly utilized and well known in the art. Housing 36 is made from biocompatible and non-thrombogenic material. Any suitable biocompatible material, such as titanium, or stainless steel, polycarbonate, or other polymers, may be employed, or alternatively a coating may be applied to a suitable substrate in order to enhance the biocompatibility of the structure.

With combined reference to FIGS. 3-5, rotor 40 is disposed within chamber 37 and has a symmetrical dual conical configuration. Rotor 40 is provided with an axis of rotation, which extends approximately along axis 14 and comprises hub 41 and several blades 42 arranged symmetrically around hub 41. Hub 41 is further utilized as a mounting base for a plurality of permanent magnets such as rotor magnets 44-44. These magnets are arranged at radially spaced locations along the axis of rotation of rotor 40, with the permanent magnets being provided at equally radially spaced locations. Matching sets of permanent magnets or electromagnetic housing magnets 55-55 are matched to rotor magnets 44 and embedded in housing 36 to cause the levitation and axial positioning of rotor 40.

An electric motor 26 is provided including an armature having windings 28, laminations 30, and multiple rotor permanent magnets 33 affixed to the pump rotor and located generally within blades 42. In this embodiment, multiple rotor permanent magnet 33 of high-energy-product magnet material, such as neodyminium iron boron magnetized is used. To avoid corrosion and provide excellent blood compatibility of the pump, all magnets and motor components are encased in titanium, which may be welded to provide a permanent seal. The space between the motor armature and the motor rotor is generally referred to as the gap between the motor windings and motor magnet. In this embodiment, some blood flows through the gap between the motor windings and motor magnet. Electromagnetic drive means are provided as at windings 28, laminations 30, and multiple rotor permanent magnets 33, with the electromagnetic drive means being, in turn, coupled to a source of electrical energy and arranged to deliver rotational driving energy to the rotor through the permanent magnets 33. The drive arrangement is, of course, commonly referred to as a brushless motor configuration and brushless motor drives are, of course, well known in the art. The rate of rotation of rotor 40 is conveniently controlled by means of the frequency of the field applied to electromagnetic windings 28, with the rate of rotation being controlled by the frequency of the applied electromagnetic field. In addition, any magnet of rotor could be used to generate a signal received by a sensor to determine the rotational speed of rotor 40 as it is common in such rotary devices. Such drives are, of course, commonly utilized and well known in the art.

Pump 32 is driven by electric motor 26 and is controlled preferably by a programmable controller 84 (shown in FIG. 11) that is capable of controlling the speed of the pump. The controller may also be auto-regulating to permit automatic regulation of the speed based upon feedback from ambient or integrated sensors monitoring parameters, such as rotational speed, pump and systemic blood flow rate, pressure of different heart chambers, pressure of different vessels, oxygenation level of blood, and heart chamber size. None of the mentioned sensors are shown in the Figures but could be easily integrated in housing 36, rotor 40, or outflow cannula 21. Any of the mentioned sensors are, of course, commonly utilized and well known in the art.

Referring to FIGS. 6A and 6B, the pump rotor 40 carries rotor permanent magnet 33, rotor magnet 44, blade(s) 42, and two wear-resistant bearings 43, located at the tip of the rotor at the center of its axis of rotation. Two static wear-resistant bearings, proximal static wear-resistant bearing 51 and distal static wear-resistant bearing 45 are mounted into the wall of proximal inflow streamlined strut 47 and distal inflow streamlined strut 46. The two static wear-resistant bearings are relatively pointed and projects into generally conical indents on the rotor surface wear-resistant bearings 43. Wear-resistant bearings 43, proximal static wear-resistant bearing 51, and distal static wear-resistant bearing 45 provide mechanical thrust bearings and limit axial and radial rotor displacement. Theoretically, wear-resistant bearings 43 never contact proximal static wear-resistant bearing 51 and distal static wear-resistant bearing 45 since rotor 40 is levitated and controlled in its axial and radial position by magnets 44 and housing magnets 55 to keep an axial and radial gap. In reality, infrequent and instantaneous contact occurs between wear-resistant bearings 43 and proximal static wear-resistant bearing 51 or distal static wear-resistant bearing 45 when sudden non-symmetrical loading to rotor 40 occurs and overcome the magnetic force maintaining rotor 40 levitated away from proximal static wear-resistant bearing 51 or distal static wear-resistant bearing 45. Two additional sets of permanent magnets, proximal axial thrust magnet 56, distal axial thrust magnet 57, and two rotor thrust magnets 58, are included in rotor 40, proximal inflow streamlined strut 47 and distal inflow streamlined strut 46 to assist in maintaining rotor 40 from contacting proximal static wear-resistant bearing 51 or distal static wear-resistant bearing 45 during operation.

The moment of inertia of rotor 40 is minimized by positioning its mass closer to the center of gravity (or center of mass). This may be obtained by moving the mass of the rotor needed for structural integrity closer to the center, and generally as closely as possible to the rotational axis. Rotor 40 is made from biocompatible and non-thrombogenic material of construction being either similar or identical to that employed in housing 36. Wear-resistant bearings 43 and proximal static wear-resistant bearing 51 or distal static wear-resistant bearing 45 could be made from several biocompatible and non-thrombogenic materials for example PTFE, diamond, or sapphire. Rotor 40 is provided with a hollow core or fully encapsulated void area 27, with this area providing a means for controlling the relative density of the rotor body.

The clearance between the inner surface of the pumping chamber and the periphery of the rotor varies along the axial length of rotor 40. The gap is minimal between blade 42 and chamber 37, and maximal between hub 41 and pumping chamber 37. The gap between blade 42 and pumping chamber 37 determine the gap in the brushless DC motor, wherein the smaller the gap the higher electric motor 26 efficiency. The gap between hub 41 and pumping chamber 37 determine the annular area that blood flow within, the larger the area the higher the blood flow rate through the pump. The clearance between the inner surface of pumping chamber 37 and the periphery of rotor 40 at blade 42 area preferably ranges from between about 0.1 millimeter up to about 3 millimeters, with a narrower range of between about 0.5 millimeter and 1 millimeters being generally preferred. Generally, a clearance of about 0.75 millimeters is preferred. In addition, the clearance between the inner surface of the pumping chamber 37 and the periphery of rotor 40 at hub 41 area preferably ranges from between about 1 millimeter up to about 6 millimeters, with a narrower range of between about 2 millimeter and 4 millimeters being generally preferred. Generally, a clearance of about 3 millimeters is preferred.

With attention being directed to FIGS. 3 and 7, outlet port 39 of pump 32 empties in outflow cannula 21 through discharge chamber 23 which transition from a semi lunar cross section into a circular cross section to mate outflow cannula 21 as shown in FIG. 7. Pump 32, discharge chamber 23 and outflow cannula 21 form a cylindrical shape capable of passing through a tube with an inside diameter slightly larger than cannula 21 outside diameter. Cannula 21 outside diameter ranges from between about 3 millimeter up to about 60 millimeters, with a narrower range of between about 8 millimeter and 12 millimeters being generally preferred. Generally, an outside diameter of about 10 millimeters is preferred.

Cannula 21 is made from flexible, biocompatible, and non-thrombogenic material such as silicone or urethane possibly reinforced by metallic wire to enhance the kink resistance during use. Cannula 21 has a duckbill tip 19 generally made from the same material as cannula 21 but without any reinforcement to keep it flexible and soft as to not cause any trauma to the tissue it contact. In addition, cannula 21 has at least one perforation 18 in the proximity of cannula proximal opening 17 that allows blood to exit cannula 21 in case cannula proximal opening 17 becomes partially occluded by any surrounding structure or tissue. Cannula technology is known in the art and may be employed effectively in connection with the device of the present invention. Cannula 21 does not occlude the entire outflow path of either right or left ventricle; thus preserving the ability of the natural heart to eject blood directly through the aortic valve without the blood first passing through the pump.

FIGS. 7a-c illustrate a second preferred embodiment of system 10, denoted 110. The pump generally designated 132 comprises a housing 136, the interior of which defines pumping chamber 137. Pump 132 having dual inlets, distal inlet 134 and proximal inlet 135, and an outflow cannula 121 to provide a conduit for blood energized by pump 132 to cross from the left atrium 120 into aorta 116 across aortic valve 113. Pump 132 is preferably a centrifugal rotary pump; either an axial type or a centrifugal type could be interchanged. In either case, pump 132 is sufficiently small to be implanted percutaneously through a main artery or vein such as the femoral artery or vein in the groin area of the patient, without the need for major invasive surgery. With attention being directed to FIG. 7c the inner periphery 138 of housing 136 is the outer periphery of the chamber 137. As is clear from the views of FIGS. 7a and 7c, housing 136 and chamber 137 shares a central axis that extends along axis 114 as set forth in FIG. 7c. Housing 136, and accordingly chamber 137, is provided with a pair of inlets, distal inlet 134 and proximal inlet 135, along with a pair of outlets, distal outlet port 139 and proximal outlet port 150. Inlet ports 134 and 135, collectively, define the inlets to chamber 137, while outlet ports 139 and 150, collectively, define chamber outlet 137. Inlet ports 134 and 135 are arranged coaxially with the chamber, that is, along axis 114, with the inlet ports being arranged in oppositely disposed relationship to chamber 137.

Two inflow stator, proximal inflow streamlined strut 147 and distal inflow streamlined strut 146, projects into the two pump inlets and act as a hydraulic stator to condition blood flow into pump 132. In addition, two protective cages, proximal protective cage 148 and distal protective cage 149, protect surrounding tissue from entering the pump or from pump suction against a relatively flat surface. Distal and proximal outlet ports 139 and 150 are axially, and possibly radially, spaced to match the position of distal and proximal blade set 153 and 152 respectively. In addition, distal and proximal outlet ports 139 and 150 empty into at least one discharge chamber 123. Alternatively, a dual discharge chamber 123 system could be used, wherein each outlet port, outlet ports 139 and 150, empties into a separate discharge chamber 123, wherein each discharge chamber 123 is separate from the other discharge chamber 123 empties into outflow cannula 121. Outlet port 139 and outlet port 150 are formed from a multitude of outflow channel 131 (similar to the one shown in FIG. 4c), which empties into discharge chamber 123. Successive outflow channels 131 are separated by hydraulic stator 129, which is designed to direct blood from chamber 37 into discharge chamber 23. Alternatively, outlet port 139 and outlet port 150 could be a tangential opening (not shown) between chamber 137 and discharge chamber 123 that approach the design of the volute of a centrifugal pump. Volute design is, of course, commonly utilized and well known in the art. Housing 136 is made from biocompatible and non-thrombogenic material. A suitable biocompatible material titanium, or stainless steel, polycarbonate, or other polymers may be employed, or alternatively a coating may be applied to a suitable substrate in order to enhance the biocompatibility of the structure.

With continued attention being directed to FIGS. 7a-c, rotor 140 is disposed within chamber 137 and has a dual conical configuration. Rotor 140 is provided with an axis of rotation, which extends approximately along axis 114 and comprises hub 141 and at least two blade sets, proximal blade set 152 and distal blade set 153. Each blade set comprise several blades 142 arranged symmetrically around hub 141. Hub 141 is further utilized as a mounting base for a plurality of permanent magnets such as rotor magnets 144-144. These magnets are arranged at radially spaced locations along the axis of rotation of rotor 140, with the permanent magnets being provided at equally radially spaced locations. Matching sets of permanent magnets or electromagnetic housing magnets 155-155 are matched to rotor magnets 144-144 and embedded in housing 136 to cause the levitation, axial positioning, and/or rotation of rotor 140. Alternatively, some or all of rotor magnets 144-144 and matched housing magnets 155-155 could also be embedded in blades 142 and housing 136 (not shown). Referring to FIG. 7c, electric motor 126 includes an armature having windings 128, laminations 130, and multiple rotor permanent magnets 133 affixed to the pump rotor and located generally between proximal blade set 152 and distal blade set 153. In this embodiment, multiple rotor permanent magnet 133 of high-energy-product magnet material, such as neodyminium iron boron magnetized is used. To avoid corrosion and provide excellent blood compatibility of the pump, all magnets and motor components are encased and permanently sealed. The space between the motor armature and the motor rotor is generally referred to as the gap between the motor windings and motor magnet. In this embodiment, some blood flows through the gap between the motor windings and motor magnet.

Electromagnetic drive means are provided as at windings 128, laminations 130, and multiple rotor permanent magnets 133, with the electromagnetic drive means being, in turn, coupled to a source of electrical energy and arranged to deliver rotational driving energy to the rotor through the permanent magnets 133. The drive arrangement is, of course, commonly referred to as a brushless motor configuration and brushless motor drives are, of course, well known in the art. The rate of rotation of rotor 140 is conveniently controlled by means of the frequency of the field applied to electromagnetic windings 128, with the rate of rotation being controlled by the frequency of the applied electromagnetic field. In addition, any magnet of rotor could be used to generate a signal received by a sensor to determine the rotational speed of rotor 140 as it is common in such rotary devices. Such drives are, of course, commonly utilized and well known in the art. As mentioned, pump 132 is driven by electric motor 126 and is controlled preferably by a programmable controller 84 that is capable of controlling the speed of the pump. The controller may also be auto regulating to permit automatic regulation of the speed based upon feedback from ambient sensors monitoring parameters, such as rotational speed, blood flow rate, pressure of different heart chambers, pressure of different vessels, oxygenation level of blood, and heart chamber size.

Referring to FIG. 7b-c, the pump rotor 140 carries rotor permanent magnet 133, rotor magnet 144, blade(s) 142, and two wear-resistant bearings 143, located at the tip of the rotor at the center of its axis of rotation. Two static wear-resistant bearings, proximal static wear-resistant bearing 151 and distal static wear-resistant bearing 145 are mounted into the wall of proximal inflow streamlined strut 147 and distal inflow streamlined strut 146. The two static wear-resistant bearings are relatively pointed and projects into generally conical indents on the rotor surface wear-resistant bearings 143. Wear-resistant bearings 143, proximal static wear-resistant bearing 151, and distal static wear-resistant bearing 145 provide mechanical thrust bearings and limit axial and radial rotor displacement. Theoretically, wear-resistant bearings 143 never contact proximal static wear-resistant bearing 151 and distal static wear-resistant bearing 145 since rotor 140 is levitated and controlled in its axial and radial position by magnets 144 and housing magnets 155 to keep an axial and radial gap. In reality, infrequent and instantaneous contact occurs between wear-resistant bearings 143 and proximal static wear-resistant bearing 151 or distal static wear-resistant bearing 145 when sudden non-symmetrical loading to rotor 140 occurs and overcome the magnetic force maintaining rotor 140 levitated away from proximal static wear-resistant bearing 151 or distal static wear-resistant bearing 145. Two additional sets of permanent magnets, proximal axial thrust magnet 156, distal axial thrust magnet 157, and two rotor thrust magnets 158, are included in rotor 140, proximal inflow streamlined strut 147 and distal inflow streamlined strut 146 to assist in maintaining rotor 140 from contacting proximal static wear-resistant bearing 151 or distal static wear-resistant bearing 145 during operation. Those thrust bearing, proximal axial thrust magnet 156, distal axial thrust magnet 157, and two rotor thrust magnets 158, are positioned with their polarity as shown in 7c to form a force that repel wear resistance bearing 143 from its matching static wear-resistant bearing in order to reduce contact and wear.

Rotor 140 moment of inertia is minimized by positioning its mass closer to the center of gravity (or center of mass). This may be obtained by moving the mass of the rotor needed for structural integrity closer to the center, and generally as closely as possible to the rotational axis. Rotor 140 is made from biocompatible and non-thrombogenic material of construction being either similar or identical to that employed in housing 136. Wear-resistant bearings 143 and proximal static wear-resistant bearing 151 or distal static wear-resistant bearing 145 could be made from several biocompatible and non-thrombogenic materials for example PTFE, diamond, or sapphire. Rotor 140 is provided with a hollow core or fully encapsulated void area (not shown), with this area providing a means for controlling the relative density of the rotor body.

The clearance between the inner surface of the pumping chamber and the periphery of the rotor varies along the axial length of rotor 140. The gap is minimal between blade 142 and chamber 137, minimal between rotor 140 and chamber 137 in electric motor 126 area, and maximal between hub 141 and pumping chamber 137. The gap between blade 142 and pumping chamber 137 affect the hydraulic efficiency of the pump. The gap between rotor 140 and pumping chamber 137 in electric motor 126 area determine the gap in the brushless DC motor, wherein the smaller the gap the higher electric motor 126 efficiency. The gap between hub 141 and pumping chamber 137 determine the annular area that blood flow within, the larger the area the higher the blood flow rate through the pump. The clearance between the inner surface of pumping chamber 137 and the periphery of rotor 140 at blade 142 area preferably ranges from between about 0.1 millimeter up to about 3 millimeters, with a narrower range of between about 0.5 millimeter and 1 millimeters being generally preferred. Generally, a clearance of about 0.75 millimeters is preferred. Similarly, the clearance between the inner surface of pumping chamber 137 and the periphery of rotor 140 at electric motor 126 area preferably ranges from between about 0.1 millimeter up to about 3 millimeters, with a narrower range of between about 0.5 millimeter and 1 millimeters being generally preferred. Generally, a clearance of about 0.75 millimeters is preferred. In addition, the clearance between the inner surface of the pumping chamber 137 and the periphery of rotor 140 at hub 141 area preferably ranges from between about 1 millimeter up to about 6 millimeters, with a narrower range of between about 2 millimeter and 4 millimeters being generally preferred. Generally, a clearance of about 3 millimeters is preferred.

Distal outlet port 139 and proximal outlet port 150 of pump 132 empty in outflow cannula 121 through discharge chamber 123 which transition from a semi lunar cross section into a circular cross section to mate outflow cannula 121 as shown in FIG. 7b. Pump 132, discharge chamber 123 and outflow cannula 121 form a cylindrical shape capable of passing through a tube with an inside diameter slightly larger than cannula 121 outside diameter. Cannula 121 outside diameter ranges from between about 3 millimeter up to about 60 millimeters, with a narrower range of between about 6 millimeter and 12 millimeters being generally preferred. Generally, an outside diameter of about 8 millimeters is preferred. Cannula 121 is made from flexible, biocompatible, and non-thrombogenic material such as silicone or urethane possibly reinforced by metallic wire to enhance the kink resistance during use. Cannula 121 has a duckbill tip 119 generally made from the same material as cannula 121 but without any reinforcement to keep it flexible and soft as to not cause any trauma to the tissue it contact. Cannula 121 is generally permanently curved to match the anatomy of the heart. This curvature will lessen any force the cannula exerts on the heart anatomy or valves in contact with the cannula. In addition, cannula 121 has at least one perforation 118 in the proximity of cannula proximal opening 117 that allows blood to exit cannula 121 in case cannula proximal opening 117 becomes partially occluded by any surrounding structure or tissue. Cannula technology is known in the art and may be employed effectively in connection with the device of the present invention. Cannula 121 does not occlude the entire outflow path of either right or left ventricle; thus preserving the ability of the natural heart to eject blood directly through the aortic valve without the blood first passing through the pump.

Now referring to the method for implanting system 10. The method described will details the placement of system 10 in the left ventricle with cannula 21 placed across the aortic valve into the aorta wherein pump 32 is removing blood from the left ventricle 11 to be discharged through cannula 21 into the aorta 16. This description is used as an example only to detail the method that could be used to place system 10 in other heart chamber to pump blood from different part of the heart to other parts of the circulatory system. It will be appreciated, of course, that various modifications may be made in the preferred embodiment illustrated above, and these modifications may be made without actually departing from the spirit and scope of the present invention.

FIGS. 8a-c illustrate an introducer system 60 of the present invention comprising dual soft and radio-opaque introducers, first curve introducer 61 and second curve introducer 62. First curvature introducer 61 comprise a tubular section, main introducer 63, with proximal opening 64 and distal opening 65 in communication with each other by main lumen 69, dilator 72, occluding balloon 66 attached to the outer surface of main introducer 63, and inflation lumen 67 (not shown) fully encapsulated into the wall of main introducer 63. Inflation lumen 67 is in communication with the internal space of occluding balloon 66 and first curve introducer 61 proximal end 68. Inflation lumen 67 is used to fill or evacuated fluid to cause the inflation or deflation of occluding balloon 67. Occluding balloon 67 serves two main functions: to occlude any perforation first curve introducer 61 creates; and to fixate first curve introducer 61 from advancing past occluding balloon 66 though a perforation. Dilator 72 is sized to freely move through first curve introducer 61 and to freely slide over catheter 71 whith minimal interference. Catheter 71 is a typical catheter commonly used in catheterization procedure and range in diameter from 1 mm to 4 mm in diameter.

First curve introducer 61 consists of a 24 inch (but can vary in range from 17 inches to 37 inches) long radiopaque thin wall polyurethane tube with a tapered tip at its distal end. The first curve introducer 61 tube size can vary from 14 French to 44 French. First curve introducer 61 is coated on both sides with a lubricating agent to facilitate its introduction into the patient and introduction of devices inside it. Second curve introducer 62 has a slightly smaller outside diameter than the inside diameter of first curve introducer 61 to allow second curve introducer 62 to slide freely inside first curve introducer 61. Curved tip 70 of second curve introducer 62 is soft enough to be able to temporarily straitened and advanced trough main lumen 69 of first curve introducer 61. As second curve introducer 62 curved tip 70 starts to exit distal opening 65 of first curve introducer 61 curved tip 70 will curve to take its normal curved shape. It is obvious that the use of the successive introducers first curve introducer 61 and second curve introducer 62, will allow the advancement of introducer system 60 into a complex path, such as the path from the atrial septum to the aorta in a human heart, while attempting to introduce a single introducer having the shape of introducer system 62 in one step is almost impossible.

FIGS. 9a-c illustrate an exemplary method of using the introducer system 60 to place a blood pumping system of the present invention into a patient's heart. Catheter 71 is inserted into the femoral vein using a conventional Seldinger technique using a Seldinger needle through which a guide wire (not shown) is threaded. Catheter 71 is advanced over guide wire into the femoral vein. Both the guide wire and catheter 71 are long enough to allow a substantial length to extend out of the body at the groin for manipulation even when the distal ends of the guide wire and catheter 71 are positioned in the heart. Catheter 71 assists in guiding system 60 to the right atrium of the heart under fluoroscopic guidance. Once catheter 71 is in the right atrium, the guide wire is withdrawn and a needle-guide wire assembly (not shown) is advanced into the catheter to the septum. The needle-guide wire is basically a needle-tipped guide wire that is capable to perforate the septum on contact. The fluoroscopic display of catheter 71 tip provides confirmation of proper orientation. When catheter 71 is properly positioned, the needle-guide wire assembly is advanced and the septum is pierced to create perforation 82.

Catheter 71 is then advanced into the left atrium and the appearance of red oxygenated blood from the left atrium in catheter 71 indicates the tip of catheter 71 is in the left atrium. The fluoroscopic display provides an indication of the actual location of catheter 71. As an option, a radio-opaque dye injected to further confirm the location of catheter 71. When the catheter 71 is properly positioned, the needle-guide wire assembly and catheter are withdrawn and removed. The needle-guide wire assembly has a stiffness sufficient to guide the catheter over it as well as have adequate flexibility to permit passage through the veins en route to the left atrium. Moreover, the integral configuration of the system allows the protected delivery of the needle assembly to the left atrium of the heart. The travel distance of the needle-guide wire assembly tip is limited to a short stroke distance in order to minimize the risk of damaging the wall of the left atrium after it has advanced through the septum.

The physician might replace the catheter 71 with another catheter with different tip geometry or stiffness at this time using standard catheter exchange procedures. At this point, the user will advance first curve introducer 61 (as shown in FIGS. 9a and 9b) through perforation 82. Perforation 82 might need to be expanded by dilator 72 of first curve catheter 61 or by using successively larger bullet tipped dilators (not shown) to increase the perforation diameter gradually before inserting first curved introducer 61. At this point occluding balloon 66 is inflated by filling it with a radio-opaque saline solution. Using fluoroscopic guidance first curve catheter 61 is advanced to cross mitral valve 15 into the left ventricle 11. Following, second curve introducer 62 is advanced inside first curved introducer 61 to fully deploy curved tip 70 inside the left ventricle (as shown in FIG. 9c).

At this point system 10 is prepared to be advanced inside the second curve introducer 62 to deploy cannula 21 across the aortic valve 13. One manner of securing the system 10 is shown in FIG. 10, wherein an insertion rod 75 is attached to the distal protective cage 49 using threaded nut 76. Distal protective cage 49 and threaded nut 76 have a mating threaded engagement to allow the attachment of one to the other. Insertion rod 75 is used to manipulate system 10 through second curve introducer 62 while advancing system 10 to its intended implant location. Insertion rod 75 is flexible enough to be able to travel through tortuous path while it is stiff enough to allow axial and rotational force be transmitted over its length. Insertion rod 75 is hollow and allows the passage of electric leads 77 through it.

After correct placement of system 10, insertion rod 75 is detached from distal protective cage 49 by rotation to loosen threaded nut 76 from distal protective cage 49. Following, second curve introducer 62 is retracted while keeping system 10 in place, and then first curve introducer 61 is retrieved while keeping system 10 in place. Alternatively, second curve introducer 62 could be retracted first before detaching and removing insertion rod 75. This will allow maintaining system 10 position while removing second curve introducer 62. Repair to the septum might be required after the removal second curvature introducer 62 and insertion rod 75. Many catheter-based technology are available to repair the septum without interfering with electric leads 77 function. Now system 10 is in place and ready for operation.

FIG. 11 illustrates the blood pump 10 of the present invention within an overall system, wherein (by way of example only) the electric leads 77 are tunneled under the skin and secured to implantable battery 78. The battery 78 is preferably rechargeable by an external induction source (not shown), such as an RF induction coil that may be electromagnetically coupled to the battery to induce a charge therein. The site at which electric leads 77 exit the vein could be surgically repaired to close around electric leads 77 and stop any bleeding, or electric leads could be retrieved trough a smaller side branch of the main vein used to insert system 10. The smaller side branch could be ligated and cinched around electric leads 77 to eliminate bleeding through that site. Power to the battery 78 may be recharged periodically (by way of example) through transcutaneous RF system 79 which comprise external battery pack 80, external RF coil 81, internal RF coil 82 and charging leads 83, and controller 84. Transcutaneous RF system 79, which is worn around the patient's waist, is a well-known technology in the field of permanent assist device. External battery pack 80 comprises several rechargeable batteries with service duration ranging from several hours to several days and is charged by a standard AC operated charger (not shown). It will be appreciated, of course, that various modifications may be made in the preferred embodiment illustrated above, and these modifications may be made without actually departing from the spirit and scope of the present invention. In the preferred embodiment, electric leads 77 are coated or encapsulated inside an anti-thrombogenic coating to minimize the potential for blood coagulation during long term use.

For system removal the reverse order of the implantation is used, wherein electric leads 77 are disconnected from battery 78 then inserted into first curve introducer 61, threaded nut 76, and insertion rod 75. An extension cord could be used to assure the length of electric leads is sufficient to cross insertion rod 75 entire length. First curve introducer 61 is inserted past the atrial septal perforation and insertion rod 75 is inserted through the patient's vascular system to reach protective cage 49 of system 10. Rotating insertion rod 75 will attach threaded nut 76 to protective cage 49. Pulling insertion rod 75 will remove system 10 form its location to the outside through first curve insertion rod 75. The septal perforation left by the removal of the system and first curve insertion rod 75 could be repaired by a catheter system intended for septal defect to eliminate any possible crossing of left atrial blood into the right atrium. Alternatively, in case electric leads 77 are encapsulated by tissue growth after prolonged system use, a specialized catheter is used to grasp distal protective cage 49 of system 10, electric leads 77 are cut free at the point they enter pump 32 and system 10 is pulled out through first curve introducer 61. It will be appreciated, of course, that various modifications may be made in the preferred embodiment illustrated above, and these modifications may be made without actually departing from the spirit and scope of the present invention.

FIG. 12 illustrates dual systems according to the present invention implanted in the left and right ventricle to provide bi-ventricular support. That is, a second system 10 is implanted in the right ventricle using a modified introducer system similar to introducer system 60 in concept but with modified tip curvature to match the anatomy of the right side of the heart. In addition, system 10 deployment into the right ventricle would not require perforating the atrial septum and therefore the use of the needle-guide wire assembly is not required and could be replaced by a standard guide wire. It will be appreciated, of course, that various modifications may be made in the preferred embodiment illustrated above, and these modifications may be made without actually departing from the spirit and scope of the present invention.

FIGS. 13a-c show exemplary steps involved to implant system 110 in the left atrium 120 to provide left ventricular support, wherein system 110 is advanced through introducer system 60, as described above with, with few modifications. The first main modification is to retract introducer system 60 at the time outflow cannula 121 crossed aortic valve 113 while pump 132 is still in the left atrium 120 area. AS mentioned before, the main modification to system 110 to accommodate placing pump 132 in the left atrium while outflow cannula 121 is across aortic valve 113 is the length and shape of outflow cannula 121. Outflow cannula 121 needs to be longer and shaped in a curvilinear fashion to match the curvilinear path between the left atrium 120 and the aortic valve 113. It will be appreciated, of course, that various modifications may be made in the preferred embodiment illustrated above, and these modifications may be made without actually departing from the spirit and scope of the present invention.

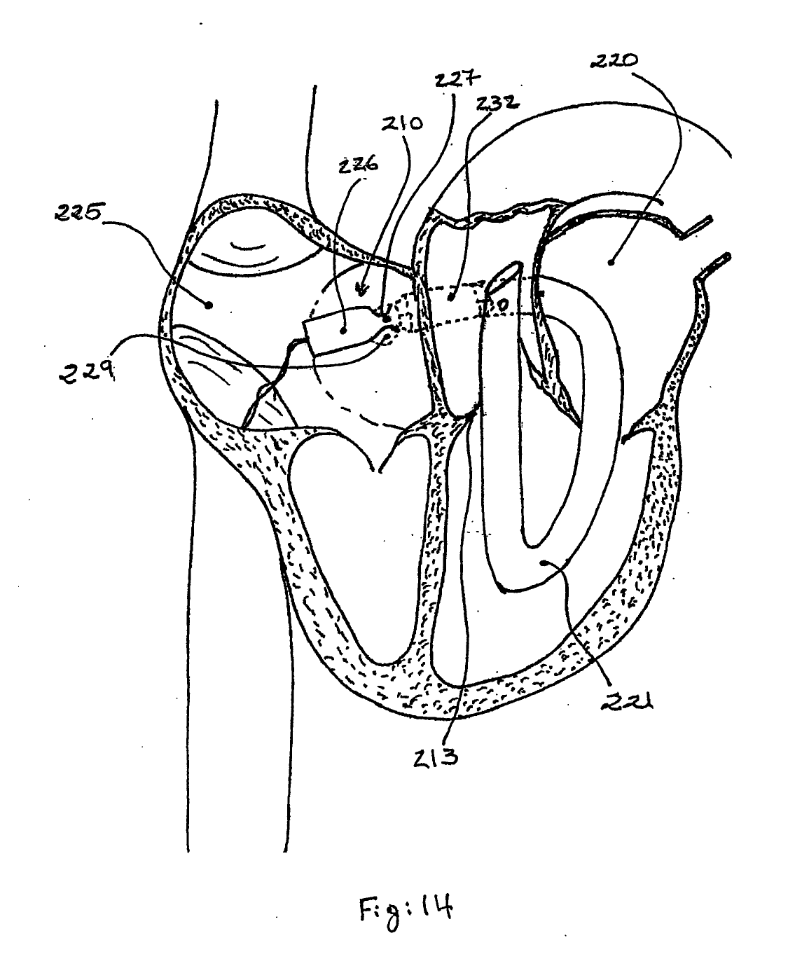

FIG. 14 shows an alternative embodiment to system 10, denoted system 210, wherein pump 232 is implanted in the left atrium 220 and electric motor 226 is in right atrium 225 with drive coupling 227 coupling electric motor 226 to pump 232. System 210 provides left ventricular support, wherein system 210 is advanced through introducer system 60, as described above with, with few modifications. The first main modification is to retract introducer system 60 at the time outflow cannula 221 crossed aortic valve 213 while pump 232 is still in the left atrium 220 area and electric motor 226 is still in the right atrium 225. The main modification to system 10 to have a mechanical drive coupling, such as a drive cable, or a magnetic drive coupling to couple electric motor 226 to pump 232 across atrial septum 229. The other modification needed to system 10 relative to system 210 in order to accommodate placing pump 232 in the left atrium while outflow cannula 221 is across aortic valve 213 is the length and shape of outflow cannula 221. Outflow cannula 221 needs to be longer and shaped in a curvilinear fashion to match the curvilinear path between the left atrium 120 and the aortic valve 213.

The main advantage of system 232 over other system is the removal of some of the system components from the left atrium to the right atrium, therefore reducing the bulkiness of the system components in the left ventricle, using the right atrium space as an added space to deploy the system, and having the two components across the atrial septum to provide a type of anchoring to the entire system. It will be appreciated, of course, that various modifications may be made in the preferred embodiment illustrated above, and these modifications may be made without actually departing from the spirit and scope of the present invention.

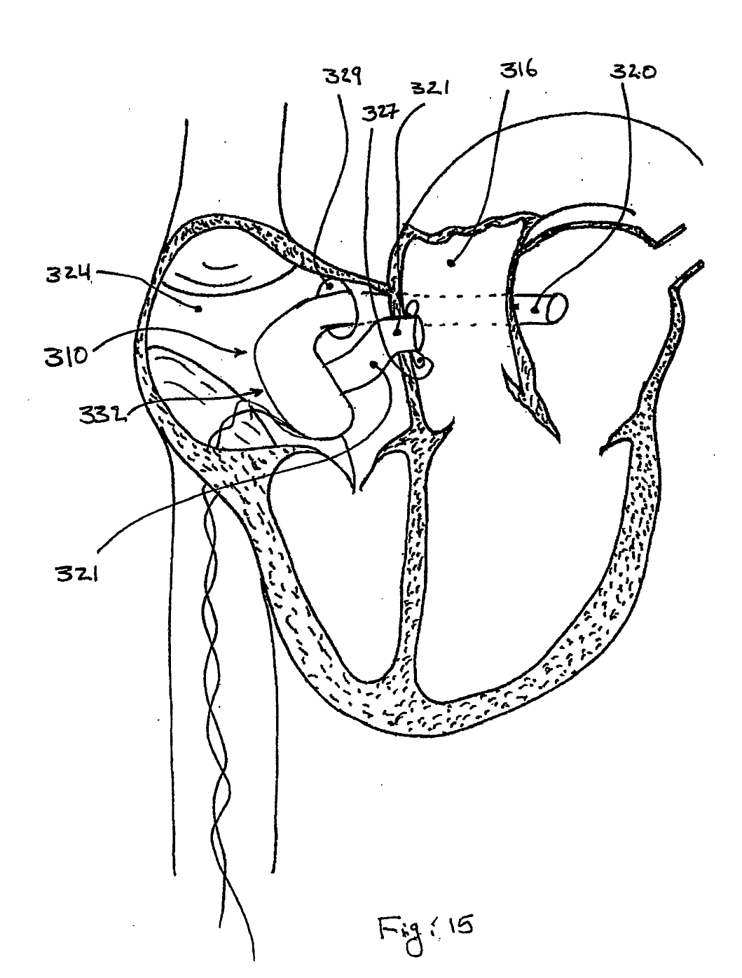

FIG. 15 shows an alternative embodiment to system 10, denoted system 310, wherein system 310 is placed in the right atrium 324 with outflow cannula 321 is placed through a perforation created between the right atrium 324 and the aorta 316; and at least one inflow cannula 320 is placed through the atrial septum 329. Outflow cannula 321 is secured to the aorta wall by means of balloon 327, wherein balloon 327 inflates by injecting fluid into balloon 327 inner space by means of a lumen embedded in outflow cannula 321 wall and in communication between balloon 327 inner space and the outside of the patient's body. The main difference between system 10 and system 310, is in the addition of at least one inflow cannula 320 to provide means for arterial blood flow into pump 332; and in the design of the outflow cannula 321 to provide means to attaches outflow cannula 321 to the aorta 316 while avoiding any leakage of arterial blood from aorta 316 to right atrium 324. It will be appreciated, of course, that various modifications may be made in the preferred embodiment illustrated above, and these modifications may be made without actually departing from the spirit and scope of the present invention.

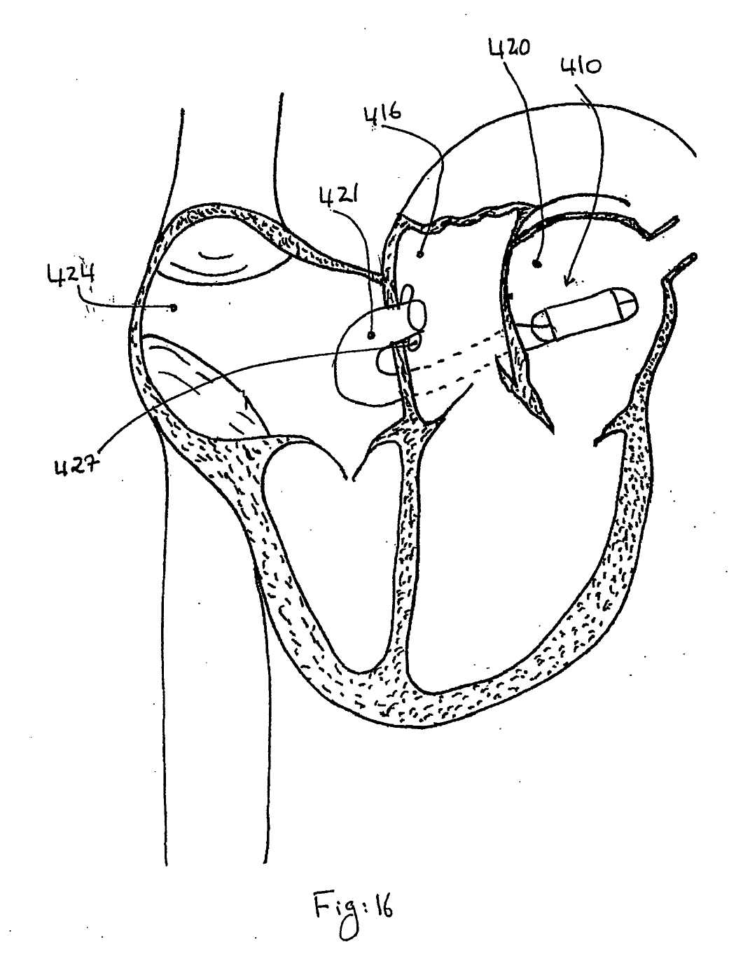

FIG. 16 shows another alternative embodiment to system 10, denoted system 410, wherein system 410 is placed in the left atrium 420 with outflow cannula 421 is placed through an atrial septal perforation and perforation created between the right atrium 424 and the aorta 416. Outflow cannula 421 is secured to the aorta wall by means of balloon 427, wherein balloon 427 inflates by injecting fluid into balloon 427 inner space by means of a lumen embedded in outflow cannula 421 wall and in communication between balloon 427 inner space and the outside of the patient's body. The main difference between system 10 and system 410, is in placing outflow cannula 421 through the atrial septum and through the right atrial wall instead of placing outflow cannula 421 through the left ventricle and the aortic valve; and in the design of the outflow cannula 421 to provide means to attaches outflow cannula 421 to the aorta 416 while avoiding any leakage of arterial blood from aorta 416 to right atrium 424. It will be appreciated, of course, that various modifications may be made in the preferred embodiment illustrated above, and these modifications may be made without actually departing from the spirit and scope of the present invention.

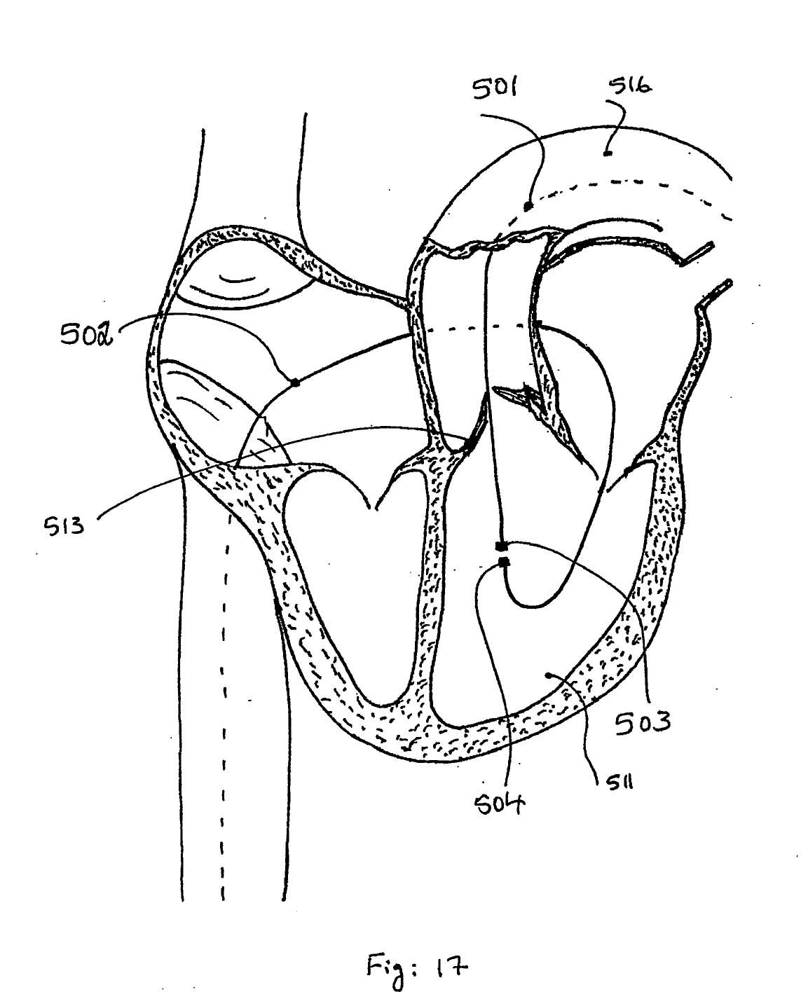

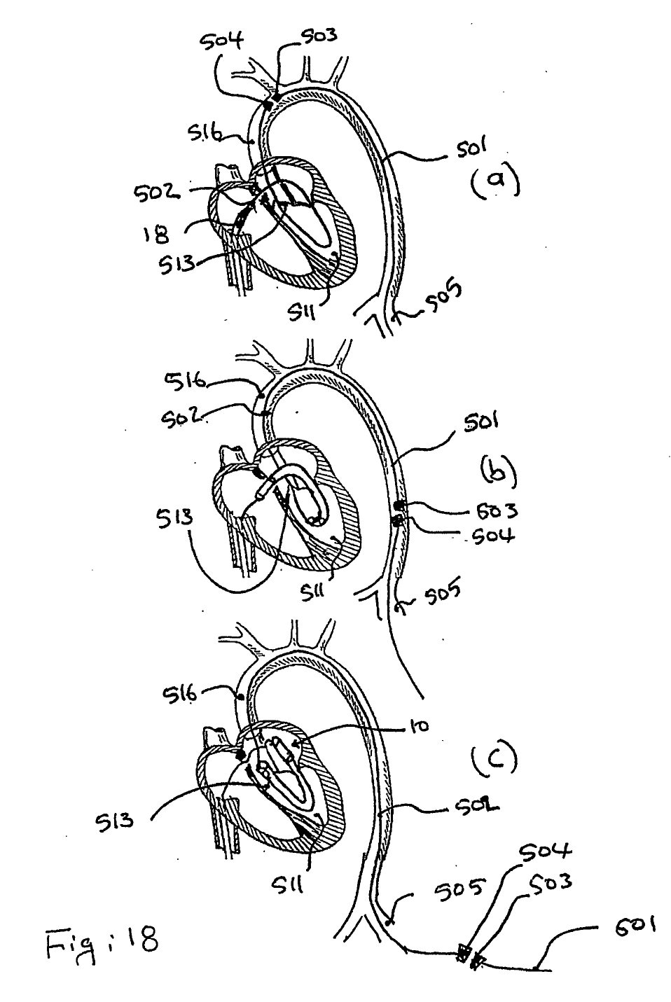

FIGS. 17 and 18a-c collectively show another alternative insertion method, wherein system 10, or any alternative embodiment, in inserted into the patient vasculature by pulling the system through the vasculature by the use of a specialized guide wire. Dual guide wire system 500 consists of two guide wires, pull guide 501 and push guide 502, wherein, both guide wires are tipped with a magnetic element. Pull guide 501 has pull magnet 503 and push guide has push magnet 504, wherein the magnet polarity is arranged as to the magnetic tip of pull guide 501 and push guide 502 attract when they are in proximity to each other.

Pull guide 501 is inserted through a vessel (for example the femoral artery), while push guide 502 is inserted through another vessel (for example the femoral vein), until pull magnet 503 and push magnet 504 meet and attract. In this fashion, they form one continuous guide wire that extends from the one vessel to another vessel that is remote from the first vessel, which would otherwise be difficult to cross if inserting one guide wire to be advanced through the full path. Of course push guide 502 will need to cross through a pre-existing or pre-formed septal perforation to allow its advancement from the venous to the arterial portion of the vasculature. Any other perforation, such as ventricular septum or vessel perforation could be used in place of the example given above to mate the two magnetic tips, pull magnet 503 and push magnet 504.

FIGS. 18a-c detail a method for inserting system 10 employing the guide wire system shown in FIG. 17. Outflow cannula 21 of system 10 is attached to the proximal end of push guide 502 by the use of a ring or a hook that attaches to perforation 18 of inflow cannula 21. At this point, pull magnet 503 and push magnet 504 are introduced into the femoral artery 505 and femoral vein respectively (not shown) and advanced to the left ventricle. Push magnet 504 is advanced through a pre-existing or a preformed septal perforation into the left atrium and then into left ventricle 511. Pull magnet 503 is advanced through femoral artery 505 into left ventricle 511 passing through aortic valve 513. After pull magnet 503 and push magnet 504 are attracted, pull wire 501 is pulled back though femoral artery 505. Push guide 502 and system 10 will be pulled into the venous system, through the atrial septal perforation, and ultimately into left ventricle 511. In addition to magnetic attraction a mechanical locking system (not shown) could be used in conjunction with pull magnet 503 and push magnet 504 to attain a higher attachment force; wherein, for example, the tip of pull guide 501 and push guide 502 could be threaded to mate when rotated opposite to each other, therefore rotating pull guide 501 and push guide 502 in opposite direction will firmly engage pull guide 501 and push guide 502 to each other. In other words, pull magnet 503 and push magnet 504 are intended to bring the distal ends of pull guide 501 and push guide 502 in proximity and rotation of pull guide 501 and push guide 502 in opposite direction will cause pull guide 501 and push guide 502 to firmly engage to each other. At this point, push wire 502 could be freed from perforation 18 of inflow cannula 21 and both pull guide 501 and push guide 503 are removed through femoral artery 505. A typical hook and release system is used at the proximal end of push guide 502 (not detailed due to its common use).

The main advantage of the above-described guide wire system is the ability to advance devices that are difficult to advance through a tortuous vasculature without the aid of such system. Pulling system 10 will definitely simplify the system insertion versus attempting to push it through the same vasculature. It will be appreciated, of course, that various modifications may be made in the preferred embodiment illustrated above, and these modifications may be made without actually departing from the spirit and scope of the present invention.

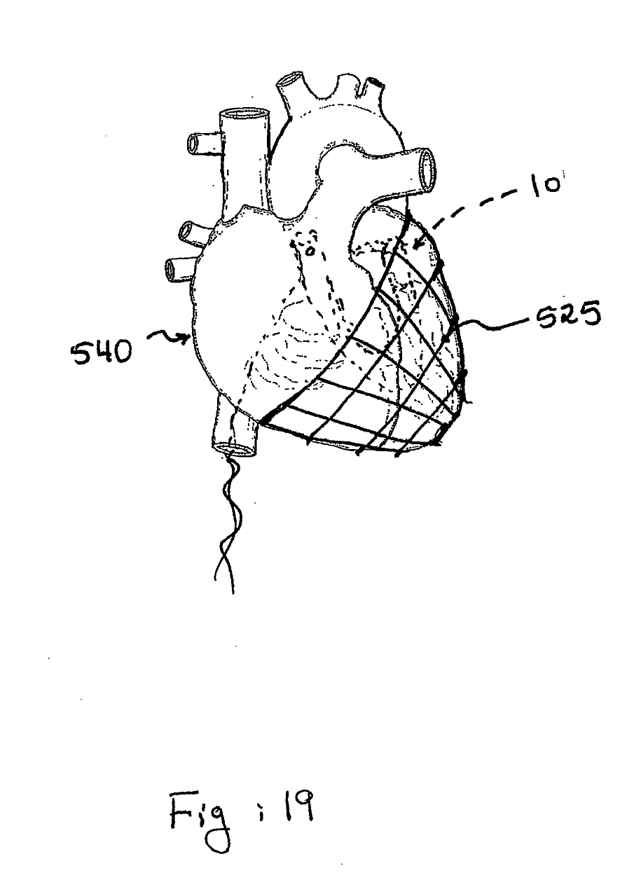

The blood pumps of the present invention may be employed with any number of known systems for facilitating or aiding the cardiac surgery or treatment. For example, with reference to FIG. 19, the blood pump 10 of the present invention may be used with a containment mesh 525 deployed around the outside surface of heart 540. Containment mesh may comprise any number of commercially available containment systems, including but not limited to, CorCap™ Cardiac Support Device developed by Acorn cardiovascular Inc of St. Paul, Minn. USA. The containment mesh may also be dynamic. In other words, the containment device may actively and continually force the ventricle's volume to decrease. For example, containment mesh 525 could be made from biocompatible elastomeric fabric that continuously has the tendency to shorten after implantation. Many similar devices that are intended to keep the heart from dilating are available on the market and use different concept. For example, a catheter based device use harpoon like pins that deploy inside the heart chamber and keep the heart from dilating. Any of these device could be used or could be slightly modified to actively shrink in case the heart size decrease. Any of these “containment” devices could be used in conjunction with system 10 (or any of the left ventricular system described above) in order to decrease the heart volume and unload the workload of the heart over an extended period of time ranging form a week to several years in order to allow the heart to shrink permanently. It is advantageous to use containment system that could be deployed percutaneously and do not require open chest surgical procedure for insertion.

In addition, the same concept described above could be used in conjunction with drugs, cellular injection of biological cellular material into the heart muscle to achieve the same ultimate effect of a “containment” system described above. For example, lab grown, autologous tissue, or synthetic engineered tissue could be injected into the diseased myocardium in conjunction with the use of a ventricular assist device, such as the different embodiments described above, in order to decrease the heart volume while allowing the cellular injections to proliferate and multiply while the heart is in a reduced volume and reduced work load, therefore, ultimately resulting in a permanent heart volume decrease and the cure of a heart hypertrophy. It will be appreciated, of course, that various modifications may be made in the preferred embodiment illustrated above, and these modifications may be made without actually departing from the spirit and scope of the present invention.

While this invention has been described in terms of a best mode for achieving this invention's objectives, it will be appreciated by those skilled in the art that variations may be accomplished in view of these teachings without deviating from the spirit, or scope of the present invention. As can be envisioned by one of skill in the art, many different combinations of the above may be used and accordingly the present invention is not limited by the scope of the appended claims.

What is claimed is:

1. A blood pump for percutaneous introduction into a patient, comprising:
a pump housing having at least two blood inlets, at least one blood outlet, and a rotor chamber extending between said two blood inlets and said blood outlet; and
a rotor disposed within said rotor chamber and operable to draw blood into said two blood inlets and direct blood out said blood outlet.

2. The blood pump of claim 1 and further, wherein said at least two blood inlets are disposed on opposing ends of said pump housing.

3. The blood pump of claim 2 and further, wherein said blood outlet is disposed generally between said at least two blood inlets.

4. The blood pump of claim 1 and further, including an outflow cannula coupled to said blood outlet.

5. The blood pump of claim 4 and further, wherein said outflow cannula is dimensioned to extend across a valve within the patient's heart.

6. The blood pump of claim 4 and further, wherein said cannula is dimensioned to extend through an opening created in the atrial septum of the patient's heart.

7. The blood pump of claim 4 and further, wherein said cannula is dimensioned to extend through an opening created in the aorta of the patient's heart.

8. The blood pump of claim 1 and further, wherein said pump housing and said rotor are equipped with cooperating magnets to position said rotor within said pump housing.

9. The blood pump of claim 1 and further, wherein said pumping housing and said rotor are equipped with thrust bearings.

10. The blood pump of claim 9 and further, wherein said thrust bearings includes a least one magnet.

11. The blood pump of claim 1 and further, including a protective cage disposed over at least one of said blood inlets.

12. The blood pump of claim 1 and further, including a control system for controlling the operation of said rotor.

13. The blood pump of claim 12 and further, wherein said control system including at least one battery coupled to an electric motor formed by magnets on said pump housing and said rotor.

14. The blood pump of claim 13 and further, wherein said battery is rechargeable.

15. The blood pump of claim 14 and further, wherein said battery is subcutaneous and said recharging is accomplished via an inductive coil.
